(19) United States
(12) Patent Application Publication
(10) Pub. No.: US 2014/0088618 A1
(43) Pub. Date: Mar. 27, 2014

(54) ELASTOMERIC AND DEGRADABLE HIGH-MINERAL CONTENT POLYMER COMPOSITES

(71) Applicants: Jie Song, Shrewsbury, MA (US); Artem Kutikov, Worcester, MA (US)

(72) Inventors: Jie Song, Shrewsbury, MA (US); Artem Kutikov, Worcester, MA (US)

(21) Appl. No.: 13/624,533

(22) Filed: Sep. 21, 2012

(51) Int. Cl. A61L 31/06; A61F 2/00

(52) U.S. Cl. CPC A61L 31/06; A61F 2/0063
USPC 606/151; 523/115

(57) ABSTRACT

The invention provides novel compositions of hydroxyapatite and block co-polymers, methods of their preparation, and uses thereof, wherein the co-polymers have degradable hydrophobic blocks and hydrophilic blocks for stable interfacing with hydroxyapatite, resulting in stable polymer-hydroxyapatite suspensions. The super-hydrophilicity, strengthened mechanical integrity, and retained structural integrity of the HA-PELA composite in aqueous environment represent major advantages over the HA-PLA composites for skeletal tissue engineering applications.

### PRIORITY CLAIMS AND CROSS REFERENCE TO RELATED APPLICATIONS

This application claims the benefit of priority from U.S. Provisional Application Ser. No. 61/538,223, filed Sep. 23, 2011, the entire content of which is incorporated herein by reference in its entirety.

### GOVERNMENT RIGHTS

This invention was made with Government support under grant nos. R01AR055615 and R01GM088678 awarded by the National Institutes of Health and under grant no. W81XWH-10-0574 awarded by the Department of Defense. The Government has certain rights in the invention.

### TECHNICAL FIELDS OF THE INVENTION

The invention generally relates to polymer compositions. More particularly, the invention relates to compositions of hydroxyapatite and block co-polymers, methods of their preparation and uses thereof, wherein the co-polymers have hydrophilic and biodegradable hydrophobic blocks for stable interfacing with HA, resulting in stable polymer-hydroxyapatite suspensions suitable for a variety of applications.

### BACKGROUND OF THE INVENTION

Significant research effort has been devoted to the development of degradable polymer/bioceramic composite materials for musculoskeletal tissue engineering. Such materials combine the tunable chemical and mechanical properties of synthetic polymers with osteoconductive yet brittle biominerals such as hydroxyapatite (HA), the principle mineral component of bone. HA provides the necessary mechanical strength, enhances the material's osteoconductivity, and serves an important source for calcium and phosphate ions. HA also plays an important role in retaining a variety of proteins on its surfaces as it has been shown to support bone cell attachment and growth factor binding and release, and to expedite healing of bone defects in vivo. (Gaharwar, et al. 2011 Biomacromolecules 12, 1641-50; Xu, et al. 2009 J. Orthop. Res. 27, 1306-11; Filion, et al. 2011 Tissue Eng. Part A 17, 503-11.) Characterized with its high stiffness and brittleness, however, HA alone is not well suited for broad orthopedic applications beyond serving as a non-weight bearing bone void filler.

To address such limitations, HA has been incorporated with synthetic polymers to form 2- or 3-dimensional, dense or porous composite scaffolds using a wide range of fabrication techniques including electrospinning, injection molding, and 3-dimensional prototyping. (Venugopal, et al. 2008 Artif. Organs 32, 388-97; Yang, et al. 2009 Acta Biomater. 5, 3295-304; Erisken, et al. 2008 Nanotechnology 19, 165302; Wang 2003 Biomaterials 24, 2133-2151; Kim, et al. 2012 Biofabrication 4, 025003; Butscher, et al. 2011 Acta Biomater. 7, 907-20.) Adequate interfacial adhesion/affinity between HA and the polymeric component is a key to achieving structural and mechanical integrity. (Supová 2009 J. Mater. Sci. Mater. Med. 20, 1201-13; Kim, et al. 2004 J. Biomed. Mater. Res. 70A, 467-479.)

A widely used fabrication technology for generating porous thin membrane scaffolds (or fibrous meshes) is electrospinning, where a grounded surface collects a charged polymer jet of nano and/or micro-sized fibers. Previously reported co-electrospinning of various polymers with hydroxyapatite suffers from a variety of limitations, such as material defects, settling of the hydroxyapatite, poor integration and brittleness, low strength and inferior surgical handling properties. Although beneficial effects occur when blending HA with hydrophilic polymers such as poly(hydroxyethyl methacrylate), for example improved toughness, elastic modulus and osteoblast adhesion, unfortunately poly(hydroxyethyl methacrylate) is not biodegradable.

Biodegradable polyesters such as poly(lactic acid) (PLA) are readily electrospinnable with established in vitro and in vivo degradation profiles. The intrinsic hydrophobicity of PLA, however, results in its poor mixing and adhesion with hydrophilic HA, making it difficult to achieve adequate structural and mechanical properties in electrospun HA-PLA composite meshes. (Supová 2009 J. Mater. Sci. Mater. Med. 20, 1201-13; Qiu, et al. 2005 Biomacromolecules 6, 1193-9; Wei, et al. Macromol. Biosci. 9, 631-8; Wang, et al. 2010 Appl. Surf Sci. 256, 6107-6112.) HA-PLA composites often exhibit inferior handling properties (e.g., brittleness) and inconsistent biological performance. Approaches for addressing the lack of interfacial adhesion include the addition of amphiphilic surfactants or modifying HA with surface-grafted polymers to improve interactions with hydrophobic polyesters. (Yang, et al. 2009 Acta Biomater. 5, 3295-304; Kim 2007 J. Biomed. Mater. Res. A, 83, 169-77; Qiu, et al. 2005 Biomacromolecules 6, 1193-9; Kim, et al. 2006 J. Biomed. Mater. Res. A 79, 643-9; D'Angelo, et al. 2012 Biomacromolecules, DOI 10.1021/bm3000716.)

Such approaches, however, often introduce additives with ill-defined or unknown biological consequences. Alternatively, HA mixing and adhesion can be achieved through favorable hydrophilic interactions. For example, blends of HA with hydrophilic polymers such as poly(hydroxyethyl methacrylate) (pHEMA) and poly(ethylene glycol) (PEG) have been reported. (Gaharwar, et al. 2011 Biomacromolecules 12, 1641-50; Song 2003 J. European Ceramic Soc. 23, 2905-2919; Song, et al. 2009 J. Biomed. Mater. Res. A. 89, 1098-107.) The PEG-HA composites exhibited remarkable mechanical properties as a result of strong interfacial adhesion between HA and the hydrophilic polymers. Unfortunately, hydrophilic pHEMA and PEG lack biodegradability and are not stable in aqueous environments without crosslinking, making them unsuitable for fabricating degradable HA-polymer composite meshes by electrospinning. Thus, an un-met need continues to exist for novel synthetic tissue scaffolds with desired structural and biological properties while exhibiting exceptional features such as scalability and ease of use. Achieving such delicate balance requires thoughtful selection and integration of building blocks of the synthetic scaffold, which remains a fundamental challenge in the design of synthetic tissue scaffolds.

### SUMMARY OF THE INVENTION

The invention provides novel compositions of hydroxyapatite and block co-polymers, methods of their preparation and uses thereof, wherein the co-polymers have hydrophilic and biodegradable hydrophobic blocks for stable interfacing with HA, resulting in stable polymer-HA suspensions. The super-hydrophilicity, strengthened mechanical integrity, and retained structural integrity of the HA-poly(ethylene glycol-co-lactic acid) (PELA) composite in aqueous environment represent major advantages over the HA-poly(lactic acid) (PLA) composites for various skeletal tissue engineering applications.

In one aspect, the invention generally relates to a composition that includes hydroxyapatite and a block co-polymer. The block co-polymer includes hydrophilic blocks and degradable hydrophobic blocks. The composition exhibits hydrophilic surface properties, elasticity and retains mechanical integrity in aqueous environment. In certain preferred embodiments, the composition possesses a stable structural interface between the co-polymer and the hydroxyapatite. In certain preferred embodiments, the composition of the invention is characterized by the properties of biodegradability, aqueous stability and eletrospinability. For example, the composition may be electrospun into fibrous composite mesh. In certain preferred embodiments, the block co-polymer is crosslinked to form a three-dimensional polymer-hydroxyapatite network. In certain preferred embodiments, the block co-polymer/HA composite is extruded into an un-crosslinked three-dimensional scaffold by rapid prototyping (or 3-D printing).

In another aspect, the invention generally relates to a medical implant that includes a composition comprising hydroxyapatite and a block co-polymer, wherein the block co-polymer comprises hydrophilic blocks and degradable hydrophobic blocks. For example, the implant may be a 3-dimensional filler for bony defects or a repair material for bone, cartilage, osteochondral, tendon or ligament damage.

In yet another aspect, the invention generally relates to a biodegradable composite scaffold prepared from a fibrous composite mesh electrospun from a suspension of hydroxyapatite with an amphiphilic block poly(ethylene glycol-co-lactic acid).

In yet another aspect, the invention generally relates to a biodegradable, three-dimensional composite scaffold prepared by crosslinking a suspension of hydroxyapatite with an amphiphilic block poly(ethylene glycol-co-lactic acid).

In yet another aspect, the invention generally relates to a biodegradable, three-dimensional composite scaffold, prepared by rapid prototyping from a suspension of hydroxyapatite with an amphiphilic block poly(ethylene glycol-co-lactic acid).

### BRIEF DESCRIPTION OF THE FIGURES

FIG. 1 shows representative GPC spectrum and peak analysis for as-synthesized PELA.

FIG. 2 shows exemplary ¹H NMR spectrum for purified PELA.

FIG. 3 shows exemplary photographs of stable PELA-HA (20% w/w HA) suspension over 1 week.

FIG. 4 shows an exemplary electrospinning setup.

FIG. 5 shows exemplary Von Kossa stain of PELA (A) PELA-HA (20% w/w HA) and (B) meshes. 100× magnification. 1 mL of Von Kossa stain (3% silver nitrate) was added to each mesh (1 cm²) and placed in a UV crosslinker for 10 min. Samples were then washed 3 times with MilliQ water and imaged under a bright-field light microscope.

FIG. 6 shows exemplary X-ray powder diffraction of PELA-HA mesh (33% w/w/ HA). Bulk samples were placed on a glass sample holder and XRD spectra were recorded at 40 kV/40 mA from 20° to 55° at a scan rate of 1°/min. Major HA peaks are at 26° and 32°.

FIG. 7 shows exemplary SEM of PELA (A), PELA-HA (33% w/w HA) (B), and PELA-HA (50% w/w HA) (C) meshes. Samples were sputter-coated with gold and imaged under high vacuum at 5 kV on a Quanta 200 FEG MKII SEM (FEI Inc.).

FIG. 8 shows exemplary EDX spectra of PELA-HA (33% w/w HA) meshes. Samples were coated with 25 Å of carbon and spectra were recorded at 10 kV.

FIG. 9 shows (A) qualitative depiction of recoverable strain for PELA-HA (33% w/w HA) mesh, before (1), during (2), and after (3) manual elongation. (B) Tensile testing of electrospun composite meshes.

FIG. 10 shows exemplary strain sweep data for PELA (A), PELA-HA (33% w/w HA) (B), and PELA-HA (50% w/w HA) (C) meshes. Mechanical testing was performed on a TA Instruments DMA Q800 dynamic mechanical analyzer under dry conditions at room temperature. Samples were cut into 5 mm×15 mm strips and ramped at 1 Hz from 10 μm to 200 μm strain amplitude.

FIG. 11 shows exemplary reaction scheme for the synthesis of PELA (1) and PLA (2). x=454, n=608, m=750.

FIG. 12 shows exemplary microstructural, surface and compositional properties of electrospun PELA-HA and PLA-HA composite scaffolds. (A) SEM micrographs of electrospun scaffolds. Scale bars=50 μm. (B) Fiber diameters (n=100, mean±standard deviation) of electrospun scaffolds as determined from SEM micrographs using ImageJ. *, p<0.05 (One-way ANOVA with Tukey post-hoc). (C) TGA plots of electrospun scaffolds with HA powder as a control. (D) Photographs (top) and bright-field microscopy images (bottom; 10× objective, scale bar=100 μm) of von Kossa-stained electrospun PELA-HA composites.

FIG. 13 shows exemplary surface, mechanical and microstructural property changes of electrospun PELA-HA and PLA-HA composite scaffolds upon hydration. (A) Water contact angle (n=5) of as-spun and lyophilized scaffolds following water equilibration. #, water fully absorbed (˜0°). n.a., no contact angle obtained due to scaffold shrinkage. * p<0.05 (One-way ANOVA with Tukey post-hoc). (B) Storage modulus (n=3) of dry and hydrated scaffolds at 37° C., 0.02% strain, 1 Hz. * p<0.05 (One-way ANOVA with Tukey post-hoc). (C) SEM micrographs of scaffolds after 1-week incubation in PBS (pH 7.4, Ca²⁺/Mg²⁺-free) at 37° C. Scale bar: 50 mm.

FIG. 14 shows exemplary rMSC attachment and early proliferation on electrospun PELA-HA and PLA-HA scaffolds (n=3) as determined by MTT cell viability assay at 24 and 96 h after initial cell seeding. * p<0.05 (One-way ANOVA with Tukey post-hoc).

FIG. 15 shows exemplary effect of scaffold composition (chemistry and HA content) on lineage commitment of rMSC under un-stimulated culture condition as determined by qPCR.

FIG. 16 shows exemplary stability of HA (33% w/w) suspension in 1:4 dimethylformamide/chloroform solution of PELA (A) vs. PLA (B) over 1 week. Visible settling/aggregation of HA to the bottom of the HA-PLA suspension was observed as early as day 1 while the HA remained well-dispersed in HA-PELA suspension for days.

FIG. 17 shows exemplary degradation of HA-PELA and HA-PLA scaffolds in PBS at 37° C. as monitored by mass reduction over 12 weeks.

FIG. 18 shows exemplary macroporous 3-D PELA and HA-PELA composites prepared by 3-D prototyping. (A) Top view (top) and side view (bottom) photographs of 3D printed PELA, 10% and 25% HA-PELA scaffolds fabricated using a MakerBot 3D printer. Scale bar=1 mm. (B) Computer-assisted design (CAD) of the macroporous scaffold.

FIG. 19 shows exemplary scanning electron microscopy (SEM) images of 3D-printed 10% HA-PELA scaffold. Top view (left) and cross section (right).

### DESCRIPTION OF THE INVENTION

This invention provides a novel biodegradable composite material: a stable suspension of HA with a block copolymer of poly(ethylene glycol) and poly(lactic acid). For example, disclosed herein is an electrospun, biodegradable amphiphilic block copolymer/hydroxyapatite composite based on poly(ethylene glycol-co-lactic acid) (PELA). The block co-polymer fulfills key requirements, including HA integration, ease of processing (such as electrospinnability), aqueous stability, and biodegradability. The length of the PLA and PEG segments can be varied to modify the properties (mechanical, hydrophobicity, degradability) of the final polymer. PLA-PEG-PLA or PEG-PLA-PEG block copolymers can be synthesized depending on the application.

This novel approach combines the degradability and aqueous stability of the poly(D,L-lactic acid) (PLA) block with the HA-binding capability of the poly(ethylene glycol) (PEG) block and the electrospinability of both. Electrospun PELA-HA composites exhibit a more uniform fiber morphology than those of electrospun PLA-HA composites. The HA-PELA composites are super-hydrophilic and compliant, enabling easy cell seeding and potential surgical manipulations. Equally important, the HA-PELA composite scaffolds more readily promote early osteochondral lineage commitment while suppressing the adipogenesis of bone marrow stromal cells in a HA-dose dependent manner than the HA-PLA composites.

A key aspect of this invention is the use of a block co-polymer comprised of hydrophilic and degradable hydrophobic blocks for stable interfacing with HA, resulting in stable polymer-HA suspensions. The hydrophilic blocks are important for HA binding while the hydrophobic blocks allow for degradability and aqueous stability as well as eletrospinability. This formulation can be crosslinked into a degradable three-dimensional (3-D) scaffold for filling bony defects or repairing bone, cartilage, osteochondral, tendon or ligament (such as anterior cruciate ligament) damage. It can also be extruded into fibers to serve as a degradable suture or as a material for fused deposition modeling machines, allowing for the printing of custom scaffolds. The example below describes one format (an electrospun 2-D fibrous composite mesh) for potential application as synthetic periosteum to expedite the healing (tissue integration) of structural bone allografts or 3-D tissue engineered bone constructs. This would function through the delivery of exogenous therapeutic agents (e.g., growth factors), enriching endogenous factors, and/or enabling the attachment and differentiation of stem or progenitor cells (endogenous or exogenous) at the implant site.

In one aspect, the invention generally relates to a composition that includes hydroxyapatite and a block co-polymer. The block co-polymer includes hydrophilic blocks and degradable hydrophobic blocks. The composition exhibits hydrophilic surface properties, elasticity and retains mechanical integrity in aqueous environment. In certain preferred embodiments, the composition possesses a stable structural interface between the co-polymer and the hydroxyapatite.

“Hydrophilic surface properties” here refer to properties that are characteristic of and associated with hydrophilic surfaces, for example, water contact angle below about 100°.

By “retaining mechanical integrity in aqueous environment”, it is meant that the storage modulus of hydrated composite is comparable or better than the storage modulus of the dry composite.

The term “elasticity,” as used herein, refers to the ability of a material, when prepared in an electrospun membrane format with dimensions complying to ASTM D882-97, to undergo tensile deformations of at least about 20% (e.g., preferably at least about 50%, 75%, 100%, 150% or 200%) prior to failure.

The hydroxyapatite may be present in any suitable percentage depending on the application at hand. In certain embodiments, the hydroxyapatite is present in a weight percentage of at least 1% (e.g., at least 5, at least 10%, at least 20%, from about 20% to about 50%, from about 20% to about 60%, from about 20% to about 70%, from about 1% to about 70%).

In certain preferred embodiments, the block co-polymer includes blocks of poly(ethylene glycol) and polyesters. In certain preferred embodiments, the block co-polymer comprises blocks of poly(ethylene glycol) and poly(lactic acid).

In certain preferred embodiments, the composition of the invention is characterized by the properties of biodegradability, aqueous stability and eletrospinability. For example, the composition may be electrospun into fibrous composite mesh. In certain preferred embodiments, the block co-polymer is crosslinked forming a 3-D polymer-hydroxyapatite network. In certain embodiments, the composition is a 3-D network prepared rapid prototyping.

In certain embodiments, the composition is characterized by a shape-memory property. Shape-memory property refers to the ability of a material to return from a deformed state (temporary shape) to its original (permanent) shape induced by an external stimulus (trigger), such as a temperature change.

The invention also features an article of manufacture made from the composition disclosed herein.

In another aspect, the invention generally relates to a medical implant that includes a composition comprising hydroxyapatite and a block co-polymer, wherein the block co-polymer comprises hydrophilic blocks and degradable hydrophobic blocks.

For example, the implant may be a 3-D filler for bony defects or a repair material for bone, cartilage, osteochondral, tendon or ligament damage.

In certain embodiments, the implant is a degradable fibrous membrane wrapped around one or more structural allografts or one or more 3-D synthetic scaffolds to augment tissue repair function.

In certain preferred embodiments, the implant is biodegradable. Additionally, it is preferred that the implant is capable of supporting attachment of cells and/or supporting attachment of a biological agent. Any suitable cells may be employed depending on the desired application. In certain preferred embodiment, the cells are stem or progenitor cells. The biological agent may be a therapeutic, diagnostic or imaging agent. For example, the biological agent attached to the implant is a growth factor or an antibiotic agent.

In yet another aspect, the invention generally relates to a biodegradable composite scaffold prepared from a fibrous composite mesh electrospun from a suspension of hydroxyapatite with an amphiphilic block poly(ethylene glycol-co-lactic acid).

In yet another aspect, the invention generally relates to a biodegradable, three-dimensional composite scaffold prepared by crosslinking a suspension of hydroxyapatite with an amphiphilic block poly(ethylene glycol-co-lactic acid).

In yet another aspect, the invention generally relates to a biodegradable, three-dimensional composite scaffold, prepared by rapid prototyping from a suspension of hydroxyapatite with an amphiphilic block poly(ethylene glycol-co-lactic acid).

The present invention describes a rational block co-polymer design consisting of hydrophilic and degradable hydrophobic segments. Such a design allows for stable suspension of HA (or similar bioceramics) at high mineral concentrations be made in such a block co-polymer solution, which can be used to develop a variety of scaffold architectures, from 2-D electrospun composite fibrous meshes to 3-D composite scaffolds, all with excellent structural integration between the polymer and mineral component.

The invention simultaneously addresses several major limitations in prior biomaterial designs, including (1) the lack of degradable yet hydrophilic materials for musculoskeletal applications; (2) the lack of adequate structural integration, thus poor mechanical properties, of organic-inorganic structural composites for orthopedic applications; (3) the difficulty of incorporating high percentage of osteoconductive mineral in synthetic bone substitute without making the composites brittle.

In the present invention, the stable suspension of a high weight percentage of HA (content close to those in human bone), critical to recapitulate the structure and biological activity of bone, can be achieved without the addition of any surfactants or potentially harmful chemicals. This is due to the unique block copolymer design integrating well-chosen hydrophilic segments (with controlled molecular weight and composition) that exhibit intrinsic affinities to the HA, yielding highly extensible and tough materials even with HA content approaching 50 w/w %. Such materials are particularly suitable for load-bearing applications (e.g., tensile and compressive) such as synthetic bone grafts or ligaments. Since the material is thermoplastic and suspends the HA without crosslinking, it can be extruded for use as degradable sutures or other applications (i.e., fused deposition modeling). The polymer/HA mixture can also be electrospun into high HA concentration membranes for the augmentation of bone repair in combination with structural allografts or 3-D tissue engineered constructs. These processing features are highly desired and attractive because (1) they are inexpensive and (2) there is no need to change any standard industrial polymer processing flow.

### EXAMPLES

### Electrospun, Biodegradable Composite of HA with Block Copolymer of poly(ethylene glycol) and poly(lactic acid)

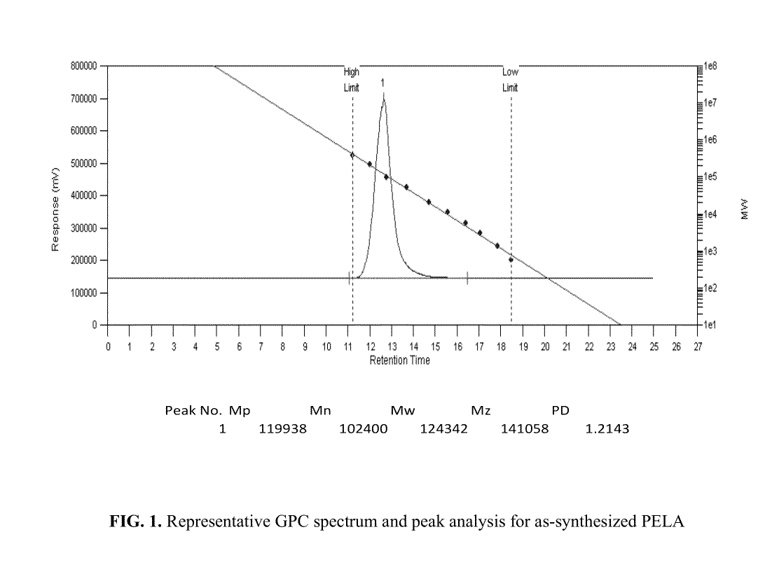

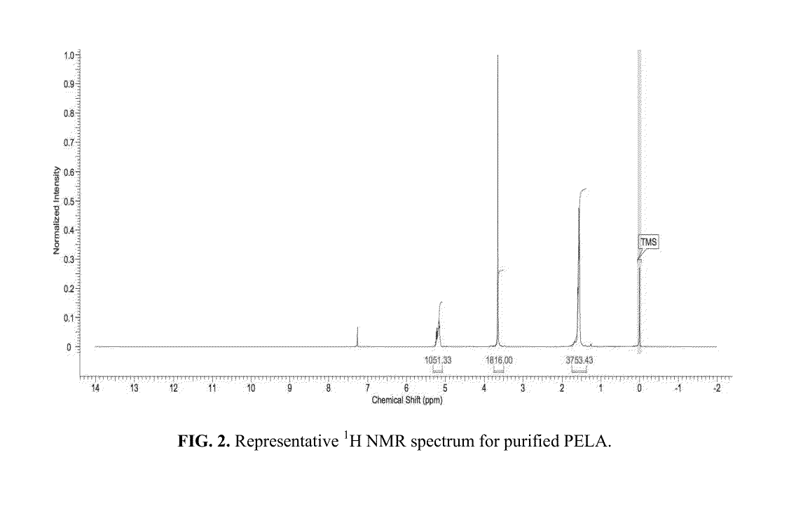

PEG (20,000 Da) was used to initiate the ring-opening polymerization of cyclic D,L-Lactide forming the block copolymer PLA₆₀₀-PEG₄₅₄-PLA₆₀₀ (PELA) or a composition of 80% w/w PLA and 20% w/w PEG. The reaction was performed at 130° C. under inert gas and catalyzed by 500 ppm of Sn(Oct)₂. The resulting polymer was characterized by gas permeation chromatography (GPC) and nuclear magnetic resonance (NMR), representative GPC and NMR data are shown in FIG. 1 and FIG. 2. The PELA was dissolved in chloroform and purified by precipitation in methanol. To prepare the electrospinning solutions, HA (from 0% to 50% w/w) was added to a 80/20 blend of chloroform/dimethylformamide and sonicated for 30 min. PELA (25% w/v) was added to the solution and dissolved by stirring and vortexing.

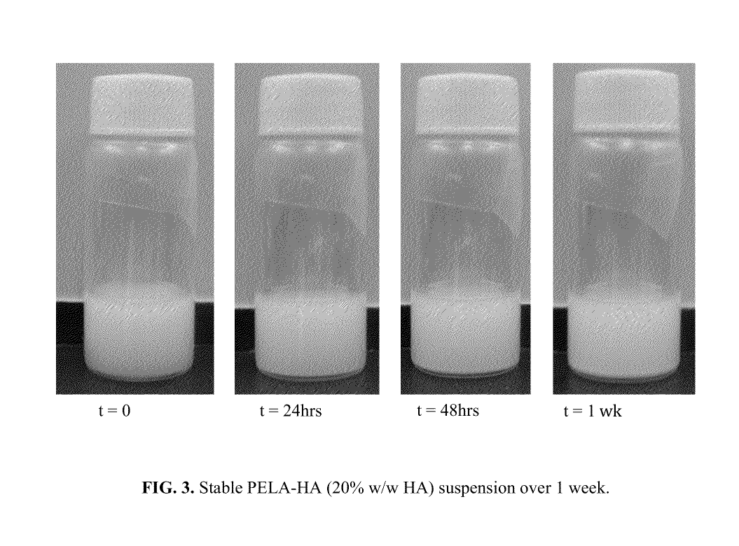

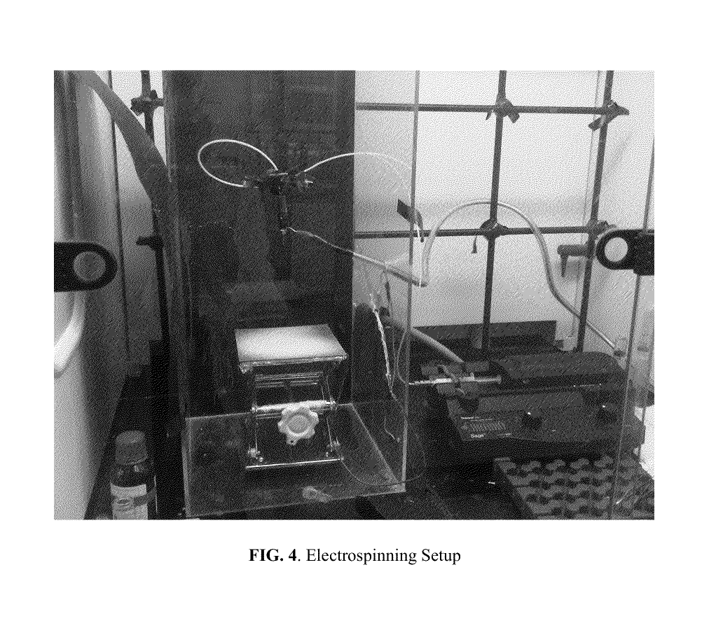

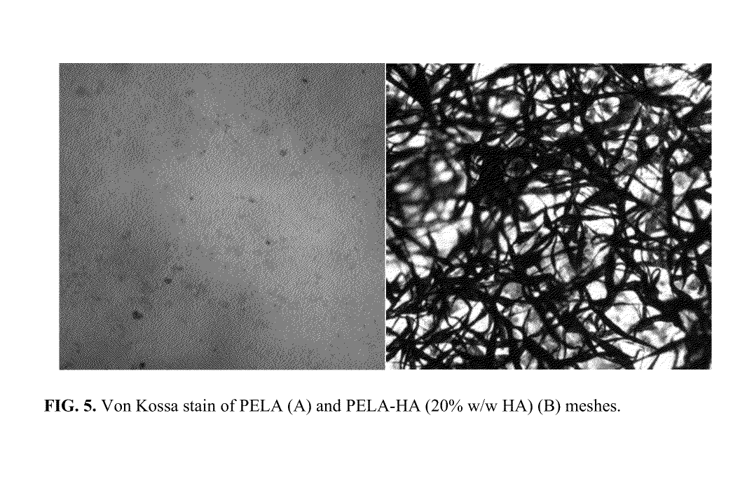

The HA remained stably dispersed in the electrospinning solution for at least 1 week with minimal settling detected which represents a major improvement over previous report (FIG. 3). The suspensions were electrospun through an 18G needle at a flow rate of 1.7 mL/hr, a voltage of 12 kV, and a working distance of 15 cm (FIG. 4). After electrospinning for 1 hr, residual solvent was removed from the meshes by drying in a vacuum oven at room temperature for 48 hr. Von Kossa staining confirmed the presence of hydroxyapatite within the electrospun fibers without non-specific HA sporadically deposited outside the fibers, supporting outstanding mineral integration with the polymer fibers (representing a major improvement over other reported methods).

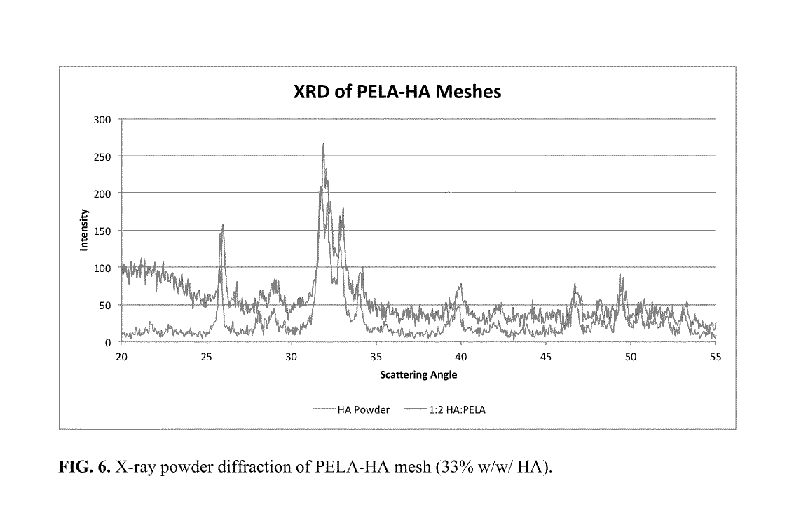

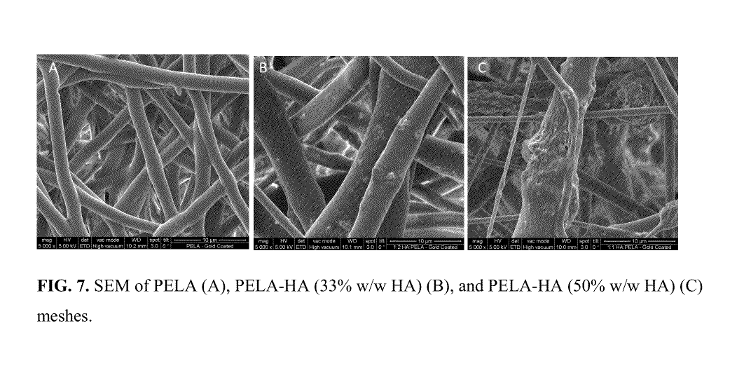

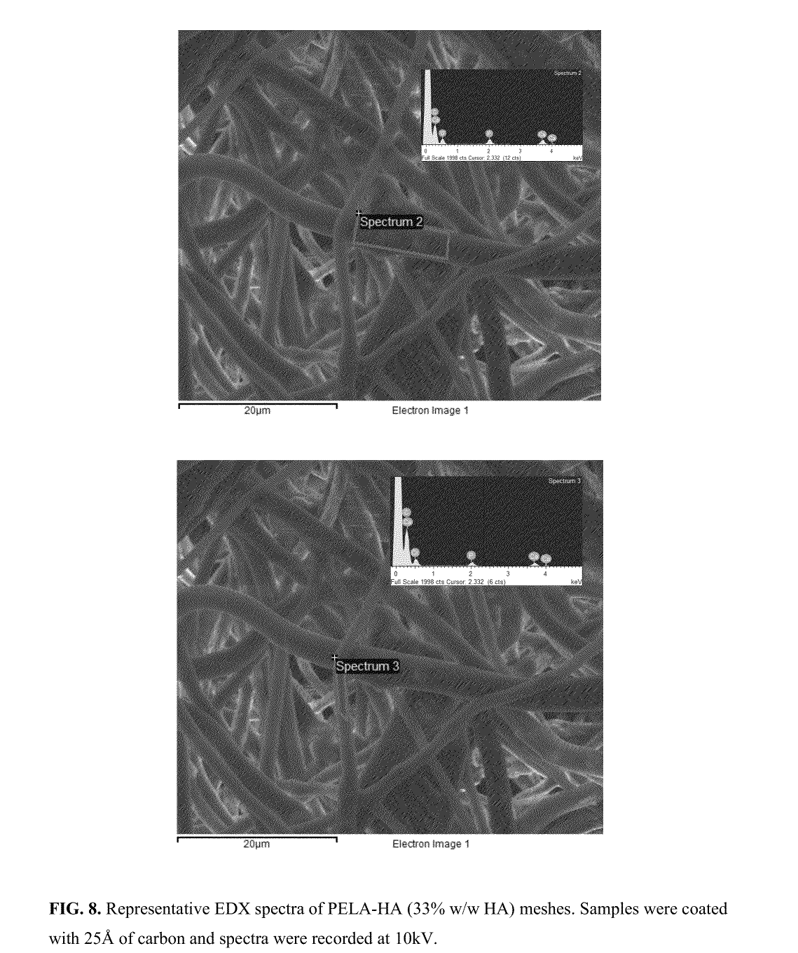

Representative bright-field images of a HA-free mesh and of a mesh containing 20% w/w HA are shown in FIG. 5. XRD confirmed no change in the crystal structure of the hydroxyapatite during the spinning process (FIG. 6). Scanning electron microscopy demonstrated that fiber dimensions were relatively uniform. Even with the incorporation of up to 50% w/w HA, HA remained embedded within the fibers with roughened fiber surfaces (FIG. 7). The presence of HA was further confirmed by energy-dispersive x-ray spectroscopy, yielding Ca/P ratios of 1.65, which is similar to the HA stoichiometric ratio of 1.67 (FIG. 8).

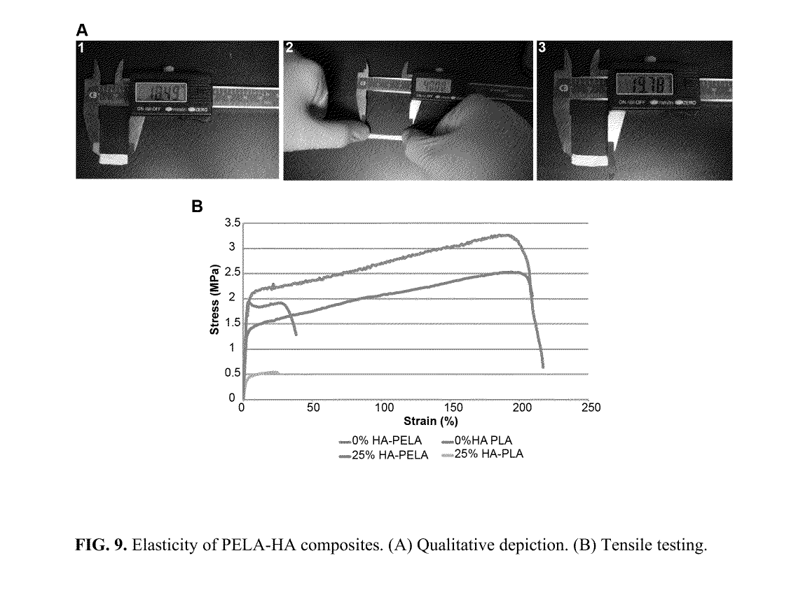

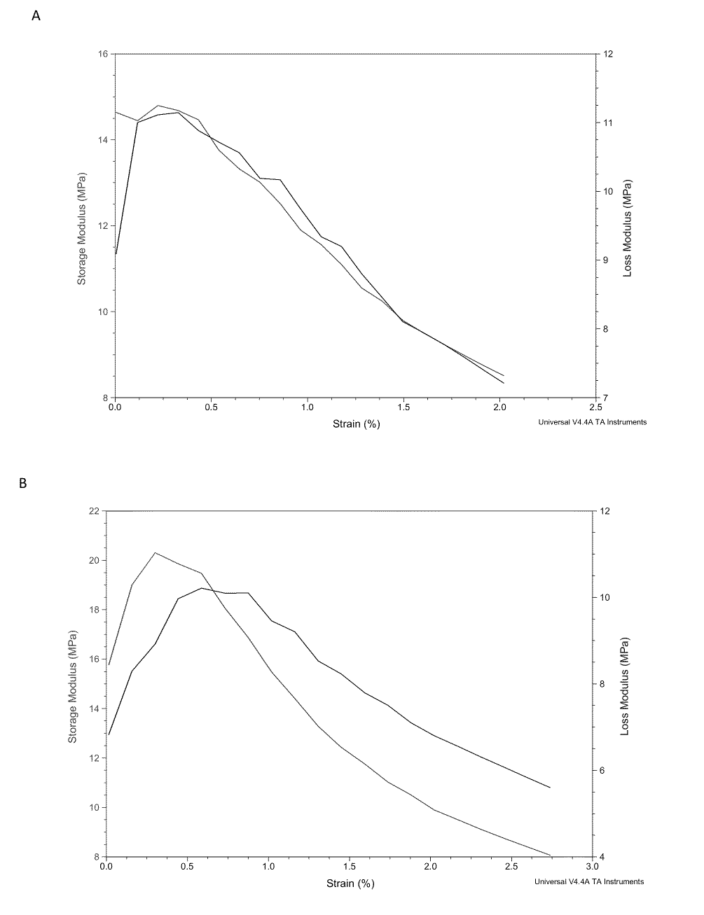

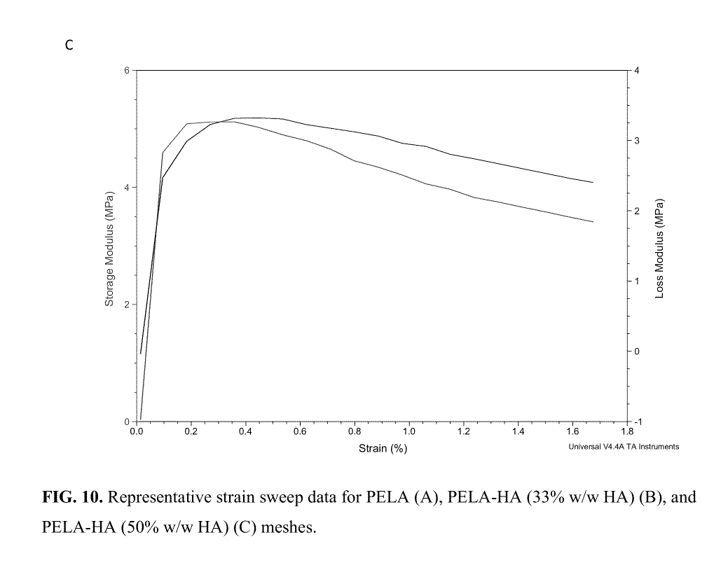

The composite meshes exhibit highly elastic properties with recoverable strains of over 100% (FIG. 9). 25% HA-PELA composite meshes exhibit ultimate tensile strains of over 200%, as opposed to 25% HA-PLA with an ultimate tensile strain below 40%. Dynamic mechanical analysis (strain sweep, 1 Hz, 0-200 μm amplitude) confirmed an increase in storage modulus with the addition of HA, further supporting good structural integration and reinforcement of the polymer matrix (FIG. 10). Meshes with 50% HA w/w exhibited a decreased storage modulus but still had high failure strains and excellent handling characteristics.

### Fabrication and Compositional Characterization of HA-PELA and HA-PLA Electrospun Composite Scaffolds

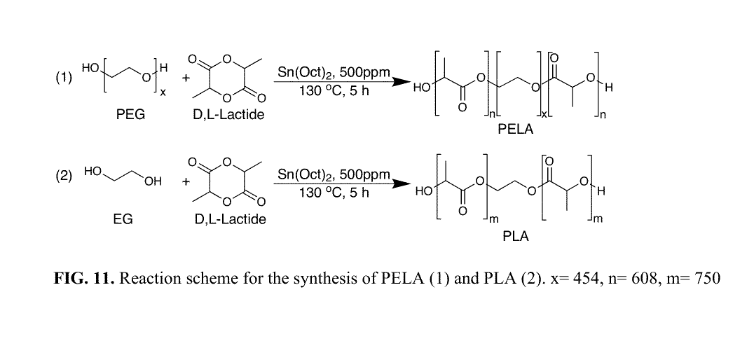

PELA and PLA control were synthesized with similar molecular weights (116,298 and 126,139 Dalton, respectively) and polydispersity (PDI=1.44) by melt ring-opening polymerization of cyclic D,L-lactide, using PEG or ethylene glycol as initiator, under the catalysis of Sn(Oct)₂ (FIG. 11). The well-controlled molecular weight and polydispersity allows us to investigate the effect of polymer chemistry on the composite scaffold properties without additional confounding factors. Racemic D,L-lactide was chosen over L-lactide due to the accelerated degradation profile of the former and the concern over undesired crystalline degradation by-products of the latter which can elicit adverse responses in vivo. (Filion, et al. 2011 Biomaterials 32, 985-91; Baroli 2009 J. Pharm. Sci. 98, 1317-75.) Differential scanning calorimetry (DSC) revealed a glass transition at 17.73° C. and 44.77° C. for PELA and PLA, respectively.

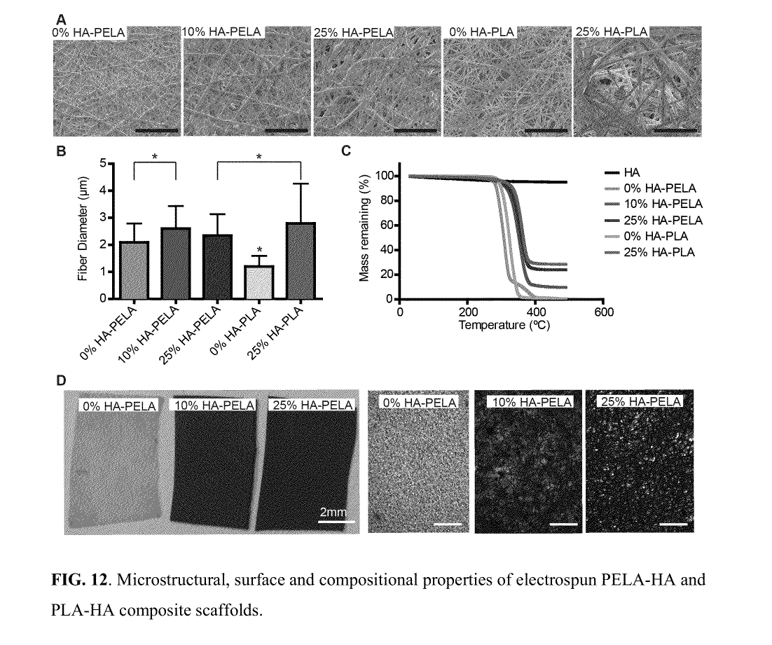

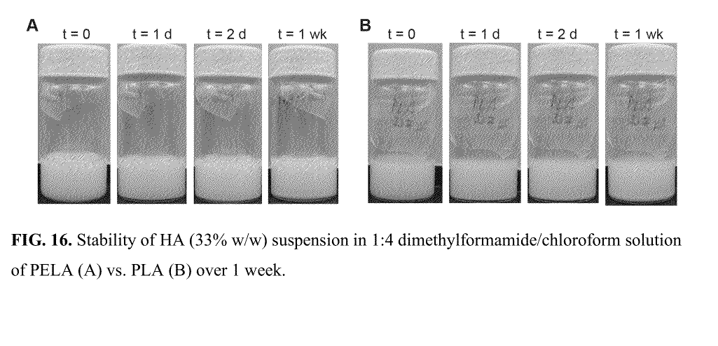

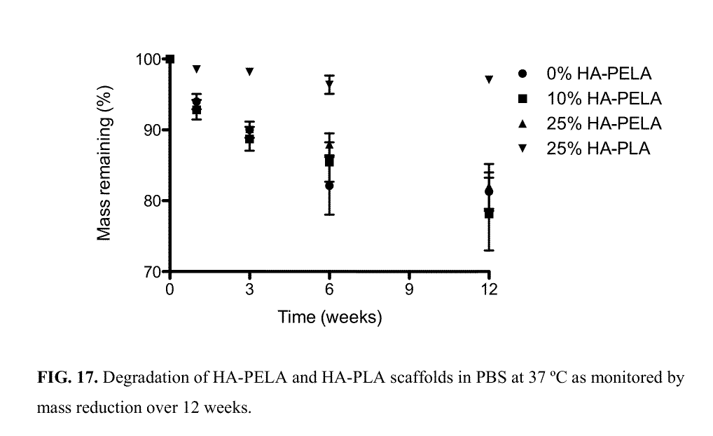

Prior to electrospinning, HA was sonicated in 1:4 dimethylformamide/chloroform to disrupt HA aggregates. PELA or PLA was then added and stirred overnight to produce a polymer/HA suspension. The PELA-HA suspension was stable for over 1 week whereas the HA tended to quickly settle from the PLA solution without immediate electrospinning (FIG. 16). The HA-PELA scaffolds were electrospun without noticeable interference from the HA component while periodic blockage of the needle tip was observed during the electrospinning of HA-PLA composite. HA-PELA and HA-PLA composite scaffolds (average thickness of 0.1-0.2 mm) with 0 to 25 wt % HA were obtained after 2 hr and dried in a vacuum oven for 48 hours to remove residual solvent. The HA-PELA composites exhibited a narrow distribution of fiber dimensions and fewer defects than HA-PLA (FIGS. 12A & B). While there was no significant difference in fiber diameter between 0% HA-PELA and 25% HA-PELA, the diameter of the 25% HA-PLA fibers increased over two-fold from 0% HA-PLA. These observations support that homogenous distribution and good structural integration of HA with the polymer was achieved with PELA, but not PLA.

Thermogravimetric analysis (TGA) was used to determine the copolymer composition and actual HA content in the electrospun composites (FIG. 12C). The 0% HA-PELA TGA curve was characterized with a transition at approximately 83% weight loss, which closely correlated with the weight percentage of PLA in the PELA scaffolds. The PLA blocks, with a lower decomposition temperature than PEG, were burnt away first. This transition was not observed in the 10 and 25% HA-PELA composite, likely due to an increase in the decomposition temperature of the composite beyond those associated with the PLA and PELA blocks upon the HA incorporation. The HA appears to have a thermal insulating effect on the scaffolds. The percentage of HA that remained after PELA thermal decomposition as determined by TGA matched precisely with their weight percentages in the electrospinning solution (10% and 25%), supporting excellent uniformity and stability of the PELA-HA suspension throughout the electrospinning. By contrast, the actual HA content in the 25% HA-PLA composite as determined by TGA is 28.4%, likely resulting from the inhomogeneous dispersion of HA with PLA and some level of settling/aggregation of HA during the electrospinning.

Homogeneous HA distribution within PELA fibrous matrix was further confirmed by von Kossa staining for calcium (FIG. 12D). (Meloan, et al. 1985 J. Histotechnol. 8, 11-13.) No HA deposition loosely associated with the electrospun fibers was detected while intense positive von Kossa stains were detected within all fibers.

### Dynamic Surface, Structural, and Mechanical Properties of HA-PELA and HA-PLA Composites as a Function of Hydration

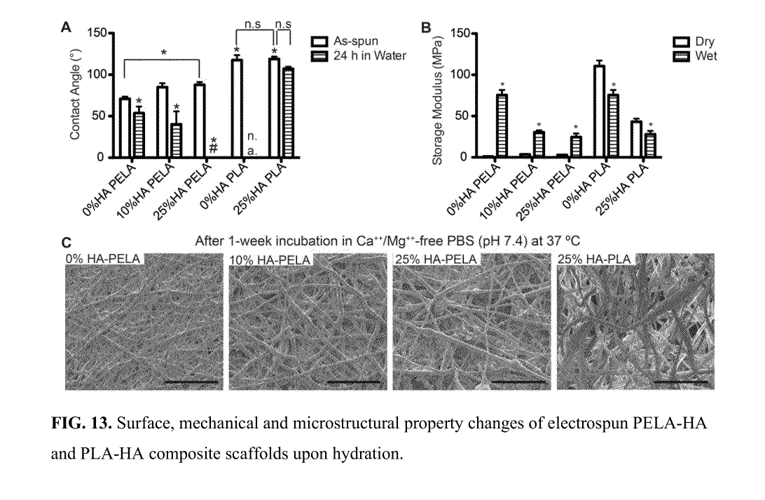

The wettability or hydrophilicity of a synthetic tissue scaffold is important for facilitating cell seeding, growth factor loading, and surgical handling. The hydrophilicity of the HA-PELA and HA-PLA electrospun composites was assessed by water contact angle measurements (FIG. 13A). As expected, the hydrophilic PEG block in PELA significantly reduced the water contact angle (increased the wettability) of the scaffolds when compared to PLA. HA incorporation slightly increased the contact angle of the vacuum-dried as-spun PELA while exerted no significant effect on the wettability of PLA. It is possible that the preferential interaction between the PEG blocks with HA has made them less exposed to the surface, contributing to the slight reduction in wettability of HA-PELA.

To examine whether and how hydration affects the structural rearrangement of the amphiphilic block copolymer as a function of HA incorporation, the scaffolds were equilibrated in water at 37° C. for 24 hr, then freeze-dried to preserve the polymer chain arrangement at the fully hydrated state (FIG. 13A). This treatment did not affect the dimensional stability but significantly reduced the water contact angle of PELA and HA-PELA composites in a HA content dependent manner, with the water contact angle on the 10% and 25% HA-PELA scaffolds dropping from >85° to around 45° and 0°, respectively. By contrast, water equilibration only reduced the water contact angle on the 25% HA-PLA scaffold by 11° C., which is expected given the limited polar groups available within the largely hydrophobic polymer that can participate in surface rearrangement in response to hydration. Contact angle measurement of the PLA could not be obtained after the water equilibration because the hydrophobic scaffold shrank dramatically. The dimensional instability of electrospun poly(D,L-lactic acid) and poly(lactic acid-co-glycolic acid) scaffolds is consistent with previous reports. (Cui, et al. 2009 Mater. Sci. Eng. C 29, 1869-1876; Jose, et al. 2009 Acta Biomater. 5, 305-15.) HA stabilizing components prevented the dramatic shrinkage of the 25% HA-PLA scaffolds upon equilibration in water.

The differential surface properties and dimensional stability changes of HA-PELA and HA-PLA upon hydration were also accompanied by drastically different mechanical property alterations. Dynamic mechanical testing was used to assess the tensile storage modulus of both dry and hydrated samples at 37° C. (FIG. 13B). The storage modulus of the dry PELA scaffolds increased from 1.1 MPa to 3.58 MPa with 10 wt % HA, then dropped slightly to 2.99 MPa with 25% HA, indicating that the HA can reinforce the scaffold with the benefit peaked at an HA content below 25%. In contrast, adding 25% HA significantly deteriorated the tensile modulus of the dry PLA scaffolds from 110 MPa to 43 MPa due to poor mixing and adhesion of the HA with the hydrophobic PLA network.

Strikingly, the 0%, 10% and 25% HA-PELA scaffolds exhibited 75-fold and 8-fold increases in storage modulus upon hydration, respectively. By contrast, the storage modulus of the electrospun PLA or HA-PLA scaffold was reduced upon hydration, which is expected due to the plasticizing effect of water.

The in vitro hydrolytic degradation behavior of the HA-PELA and HA-PLA scaffolds (n=3) was examined by monitoring mass loss upon incubation in PBS at 37° C. over a period of 12 weeks (FIG. 12). The PELA scaffolds lost ˜20% of their mass over the 12 week period, irrespective of HA incorporation content. Such a relatively rapid mass loss can be attributed to effective water penetration and the consequent hydrolytic cleavage of the polymer. (Cohn, et al. 1989 Biomaterials 10, 466-74.) By contrast, the 25% HA-PLA scaffolds only lost ˜3% of their mass after 12-week incubation likely due to ineffective water penetration throughout the hydrophobic scaffold. The structural integrity of all scaffolds examined deteriorated by 12 weeks (prone to breaking) despite the lack of more dramatic mass losses. Striking morphological differences between the scaffolds are apparent after only 1 week in PBS (FIG. 13C). The fibers of the 0% HA-PELA scaffold fused together while the fiber morphology was maintained in the 10% and 25% HA-PELA scaffolds. The degradation could be accelerated by lowering the volume of PBS (to concentrate acidic degradation products and maximize their autocatalytic effect on degradation) or by shaking (promoting water penetration).

### Impact of HA-PELA and HA-PLA Scaffolds on Lineage Commitment of Bone Marrow Stromal Cells

Mesenchymal stem cells residing in the bone marrow (MSCs) are capable of differentiating into a variety of cell types including osteoblasts, chondrocytes, adipocytes, and myoblasts. (Caplan 1991 J. Orthop. Res. 9, 641-50.) They can be readily isolated, expanded and used as a stem or progenitor cell source for musculoskeletal tissue engineering. (McCullen, et al. 2011 Curr. Opin. Biotechnol. 22, 715-20.) An effective synthetic tissue scaffold should support the attachment and guide lineage-specific differentiations of MSCs, allowing for effective regeneration of tissues of interest. Thus, the effects of HA incorporation and polymer composition on rat bone marrow stromal cell (rMSC) attachment and lineage commitment under un-stimulated culture conditions were examined.

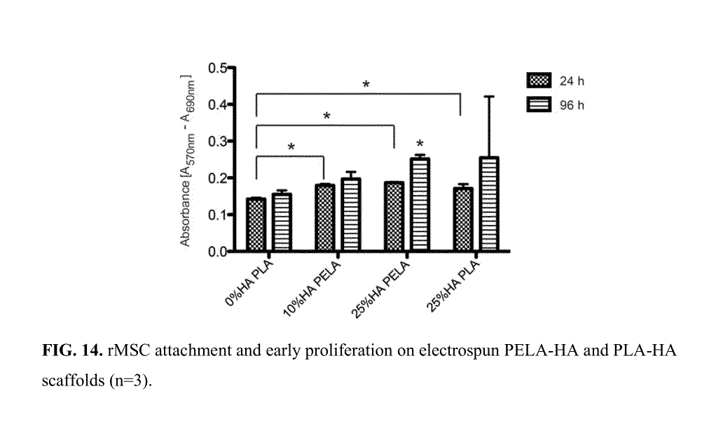

The non-fouling PEG block and the osteoconductive HA component of the HA-PELA composites are expected to exhibit opposite effects on protein and cell adhesion. Un-mineralized di-block or tri-block PELA have been used as anti-adhesion membranes, with their low protein adsorption characteristics attributed to the non-fouling PEG exposed on the surface in the aqueous environment. (Yang, et al. 2009 Acta Biomater. 5, 2467-74; Göpferich, et al. 1999 J. Biomed. Mater. Res. 46, 390-8; Lieb, et al. 2003 Tissue Eng. 9, 71-84.) By contrast, HA is known for its ability to absorb a wide range of proteins due to its dynamic surface properties (e.g., pH-dependent zeta potential) and large surface area (for HA nanocrystals) and promote cell attachment. (Gorbunoff, et al. 1984 Anal Biochem. 136, 440-445; Webster, et al. 2000 J. Biomed. Mater. Res. 51, 475-83; Lee, et al. 2010 Macromol. Biosci. 10, 173-82.) To test this hypothesis that the incorporation of HA could improve rMSC attachment on PELA in a HA dose-dependent manner, rMSCs were seeded onto the HA-PELA scaffolds with 0-25% HA (n=3) and the viability of adherent cells was quantified by MTT assay at 24 and 96 hr (FIG. 14). HA dose-dependent increases in viable adherent cells at 24 h were observed, supporting that HA promoted MSC attachment to the amphiphilic polymer. There was no significant difference in viable adherent cells at 24 h between 25% HA-PELA and 25% HA-PLA, indicating that the addition of HA effectively offset the anti-adhesion effect of the PEG component in PELA. MSCs adhered on all substrates were able to proliferate well as indicated by MTT cell viability at 96 hr, irrespective of the chemical environment and mineralization status.

The addition of HA to synthetic scaffolds has been shown to promote chondrogenesis/osteogenesis of MSCs in response to chondrogenic/osteogenic inductions in culture and skeletal tissue repair in vivo. (D'Angelo, et al. 2012 Biomacromolecules, DOI 10.1021/bm3000716; Polini, et al. 2011 PLoS One 6, e26211; Spadaccio, et al. 2009 Ann. Biomed. Eng. 37, 1376-89.) However, such an effect in a dose-dependent manner has not been easy to investigate in a reproducible manner given the inhomogeneous distribution of HA within hydrophobic degradable polymer scaffolds. In addition, most investigations interrogated the response of MSCs to specific differentiation inductions rather than their spontaneous lineage commitment under un-stimulated culture conditions as a function of HA incorporation dose in the polymer scaffolds. Such investigation is particularly lacking HA-amphiphilic polymer composites.

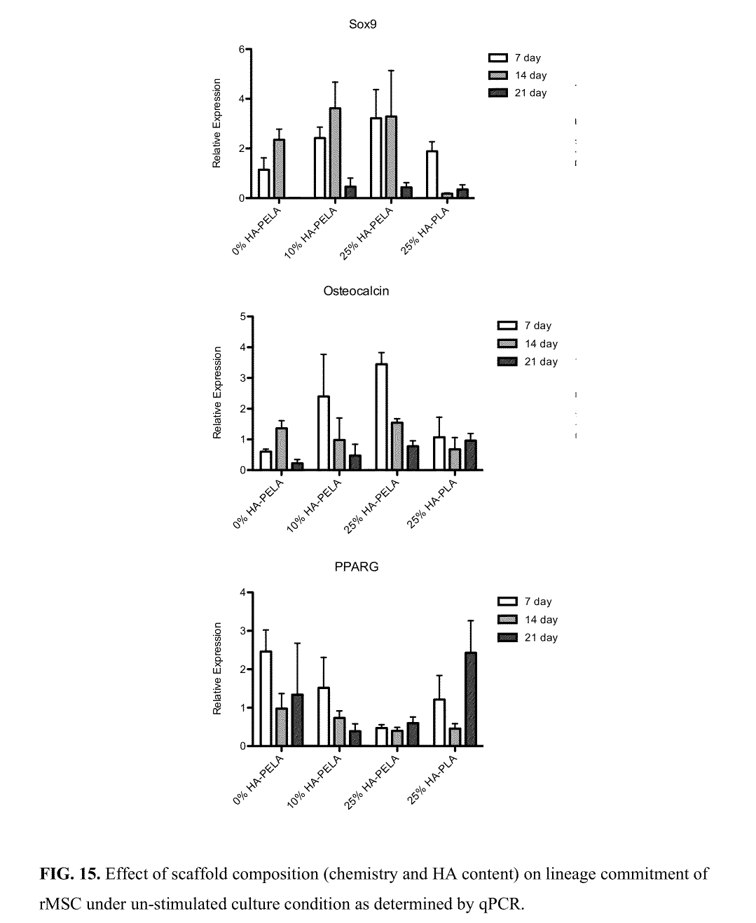

To examine the effects of dose-dependent HA incorporation in PELA and PLA on the spontaneous lineage commitment of MSCs, we cultured rMSCs on 0-25% HA-PELA scaffolds along with 25% HA-PLA control in expansion media without differentiation inducing supplements. After 7, 14, or 21 days, total RNA was isolated from cells adhered to each scaffold and the gene expression of typical osteoblast, chondrocyte, and adipocyte markers as a function of the scaffold environment and time was quantified by qPCR (FIG. 15). Data were normalized to those obtained from the rMSCs prior to seeding on the various substrates (time 0).

For the PELA scaffolds, HA incorporation resulted in dose-dependent increases in the expression of chondrogenic marker Sox9 and osteogenic marker osteocalcin as early as 7 days, with the HA dose-dependent trend persisting even when the overall expression of these markers started to decline at later time points. The decline of the overall expression of these phenotypical markers at later time points can be attributed to both the temporal nature of the expression, observed with other tissue-engineering scaffolds, and the un-stimulated culture condition that may be insufficient to drive potent and persistent expression of these markers. (Richardson, et al. 2006 Biomaterials 27, 4069-78; Shea, et al. 2003 J. Cell Biochem. 90, 1112-27; Gomes, et al. 2003 J. Biomed. Mater. Res. A 67, 87-95; Sikavitsas, et al. 2002 J. Biomed. Mater. Res. 62, 136-48.) It is worth noting that the expression of these osteochondral lineage markers was significantly higher for MSCs cultured on 25% HA-PELA scaffolds than for those cultured on 25% HA-PLA, suggesting that the osteochondro-inductive properties of HA are more effectively manifested on HA-PELA where HA were more homogeneously dispersed.

Another striking observation is that the expression of adipogenic marker PPARG significantly decreased upon the addition of HA to PELA in a dose-dependent manner at all time points examined. Furthermore, the expression of PPARG in MSCs cultured on the 25% HA-PLA scaffold is significantly higher than those cultured on the 25% HA-PELA scaffold, and the adipogenic lineage commitment promoted by the more hydrophobic HA-PELA peaked at 21 days. Overall, these data show that the PELA-HA composites promote early osteochondral lineage commitment while suppress adipogenic lineage commitment of MSCs under unstimulated culture conditions. By contrast, such effects of HA incorporation were not manifested on the PLA-HA composite, where significantly lower osteochondral gene expressions and more elevated adipocyte maker expression were observed instead.

### Rapid Prototyping (3-D Printing) of PELA and HA-PELA Filaments

Commercial polycrystalline HA (Alfa Aeser, 0, 10, or 25% w/w) was sonicated in chloroform for 30 min. prior to the addition of PELA (50% w/v) and the mixture was stirred overnight. The mixture was then poured into a Teflon mold and dried in a vacuum oven to remove residue chloroform, forming a bulk HA-PELA composite. PELA and HA-PELA was heated to 130° C. and 180° C., respectively, and drawn into 3-mm (diameter) filaments using a customized setup.

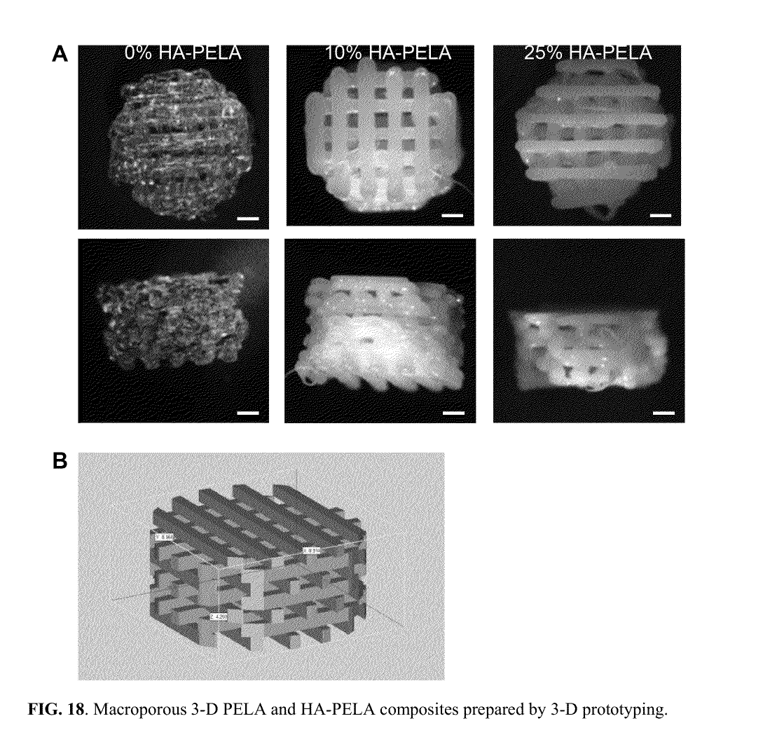

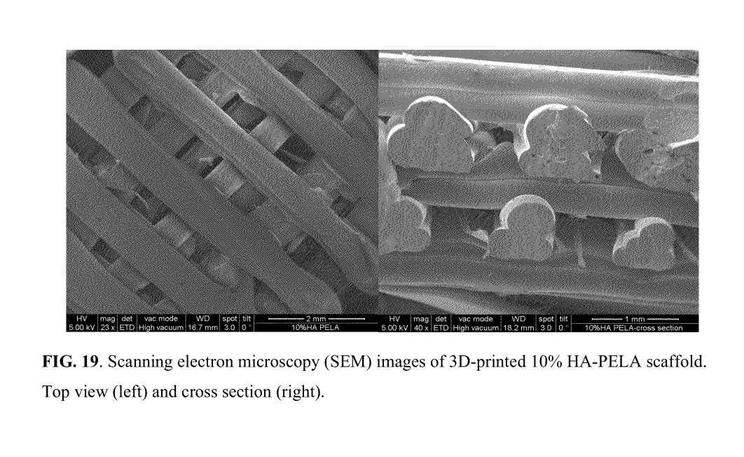

A CAD of a macroporous cylindrical scaffold was designed using 3-matics (Materialise Inc.; FIG. 18B). The CAD file was processed into g-code for 3-D printing by ReplicatorG. A MakerBot Thing-O-Matic 3D printer (MakerBot Industries) was used to rapid prototype the PELA and PELA-HA scaffolds by fused deposition modeling. The printer built the scaffolds layer-by-layer by thermal extrusion. Extruder temperatures of 140° C., 160° C., and 185° C. were used for 0% HA-PELA, 10% HA-PELA, and 25% HA-PELA scaffolds, respectively (FIG. 18A; FIG. 19).

### Experimental

Materials: 3,6-Dimethyl-1,4-dioxane-2,5-dione (D,L-lactide) was purchased from Sigma-Aldrich and purified by recrystallization twice in anhydrous toluene and dried under vacuum prior to use. Poly(ethylene glycol) (20,000 Dalton) was purchased from Fluka. Hydroxyapatite was purchased from Alfa Aesar. All other solvents and reagents were purchased from Sigma-Aldrich and used as received.

Polymer Synthesis and Characterization: Poly(ethylene glycol-co-lactic acid), PELA, was synthesized by melt ring opening polymerization. Briefly, poly(ethylene glycol) (4 g, 0.2 mmol) was heated to 100° C. in a shlenk flask and stirred under vacuum for 1 hr to remove residual water. The melt was cooled to room temperature before Tin(II) 2-ethylhexanoate (24.18 mg, 0.06 mmol) in anhydrous toluene was introduced by syringe. The toluene was removed by heating the mixture under vacuum at 100° C. for 15 min. The mixture was cooled to room temperature before D,L-lactide (17.295 g, 0.12 mol) was added under argon purge. The melt polymerization proceeded at 130° C. for 5 h under argon. Poly(lactic acid) was synthesized in the same manner with anhydrous ethylene glycol (0.87 mg, 0.1407 mol) as the initiator. Briefly, D,L-lactide (15 g, 0.1038 mol) was added to the ethylene glycol in a shlenk flask and heated to 130° C. Tin(II) 2-ethylhexanoate (21 mg, 0.05 mmol) was introduced by syringe and the melt polymerization proceeded for 5 hr. The crude PELA and PLA were dissolved in chloroform, purified by precipitation in methanol, and dried under vacuum.

Molecular weight and polydispersity of PELA and PLA was determined by gel permeation chromatography (GPC) on a Varian Prostar HPLC system equipped with two 5-mm PLGel MiniMIX-D columns (Agilent) and a PL-ELS2100 evaporative light scattering detector (Polymer Laboratories). THF was used as an eluent at 0.3 mL/hr at room temperature. Molecular weight and polydispersity was calculated based on EasiVial polystyrene standards (Agilent).

¹H NMR spectra were recorded on a Varian Mercury 400 MHz spectrometer at 298K using CDCl₃ containing tetramethylsilane as the solvent. ¹H NMR (400 MHz, CDCl₃) for PELA: δ 5.19 (m, 1126), 3.65 (m, 1816), 1.5 (m, 3921) ppm.

Thermal transitions of PELA and HA-PELA composites were determined by conventional differential scanning calorimetry (DSC) on a Q200 MDSC (TA Instruments). Samples (˜6 mg) were scanned twice from −90° C. to 250° C. (20° C./min). A constant nitrogen flow of 50 mL/min was applied. Temperature was calibrated with indium, gallium, and tin standards. T_{g} was defined as the midpoint of the inflection tangent from the second heating curve.

Electrospinning: PELA-HA and PLA-HA composite scaffolds with 0-25 wt % HA were prepared by electrospinning HA was bath-sonicated in 5-mL 1:4 dimethylformamide/chloroform for 30 min. followed by the addition of 1.25 g PELA or PLA (25% w/v). The mixture was stirred overnight at room temperature and loaded into a 5-mL syringe. A high voltage power supply (Gamma High Voltage Research) delivered a voltage of 12 kV between a 22G ejection needle and an aluminum collection plate 15 cm away. The polymer solution was fed through the needle at rate of 1.7 mL/hr with a syringe pump (Orion Sage M361, Thermo Scientific), and the fibers were collected on the aluminum collector plate. The electrospinning proceeded for 2 hr, with the collecting plate rotated every 15 min. to ensure the homogeneity of the fibrous scaffold (0.1-0.2 mm final thickness). The scaffolds were dried in a vacuum oven for 48 hr to remove any residual solvent and stored in a desiccator prior to use.

### SEM Characterization of the Scaffolds

As-spun scaffolds were sputter coated in Au (4 nm) and imaged on a Quanta 200 FEG MKII SEM (FEI Inc.) under high vacuum at 5 kV. Fiber diameter was quantified from the SEM micrographs by measuring 100 random fibers with ImageJ software (National Institutes of Health).

### Thermo-Gravimetric Analysis (TGA)

TGA was used to determine the actual percentage of HA in the as-spun scaffolds. The samples were heated at a rate of 20° C./min. from room temperature to 500° C. and the mass change was recorded on a TGA Q50 (TA Instruments). HA powder was used as a control.

### Von Kossa Staining

Von Kossa staining was carried out to confirm HA incorporation and assess HA distribution in the electrospun scaffolds. Scaffolds were cut into 5 mm×8 mm pieces and submerged in 3% silver nitrate solution. Samples were exposed to 254 nm light for 10 min. in a UV crosslinker (Stratalinker 1800, Stratagene) to develop the black stain.

### Water Contact Angle Measurement

The wettability of the scaffolds was examined by the sessile drop technique. 5-μL droplets of deionized water were deposited onto as-spun scaffolds or scaffolds freeze-dried following 24 hr equilibration in 37° C. deionized water. The water contact angle was recorded using a CAM 200 goniometer (KSV Instruments). The droplet was imaged after 30 sec. and the average contact angle from the left and right side of the drop was recorded. Five randomly selected areas per scaffold were used for each water contact angle measurement.

### Tensile Storage Modulus

The tensile storage modulus of dry and hydrated (deionized water) scaffolds (n=3) was determined on a Q800 DMA (TA Instruments). Specimens (5.3 mm×20 mm) were cut with a parallel blade cutter and loaded onto a film tension fixture with a grip separation of 10 mm. Dry and hydrated samples were applied with a 0.001 N and 0.05 N pre-load force, respectively. Samples were equilibrated at 37° C. and held isothermal for 10 min. prior to initiating 0.02% strain at a frequency 1 Hz. The 0.02% strain was chosen as it falls within the linear viscoelastic region of the scaffolds. The storage modulus at 0.02% strain was recorded.

### In vitro Hydrolytic Degradation of Scaffolds in PBS

In vitro hydrolytic degradation was determined by monitoring the mass loss of the scaffolds upon incubation in Ca²⁺/Mg²⁺-free phosphate-buffered saline (PBS) (pH 7.4) at 37° C. As-spun scaffolds were cut into 20 mm×20 mm squares (n=3 for each time point), weighed on a semi-analytical balance (XS105, Mettler Toledo) and placed in conical tubes containing 20 mL of PBS and incubated at 37° C. for up to 12 weeks. At each time point, 3 specimens were removed, washed 3 times with deionized water, and lyophilized. The mass of each retrieved and dried specimen was recorded. The morphology of the degraded scaffolds was examined by SEM as described above.

### Rat Bone Marrow Stromal Cell Attachment and Proliferation

Rat bone marrow stromal cells (rMSCs) were isolated from the long bones of a 4-week old male Charles River SASCO SD rat as previously described. Briefly, whole bone marrow was flushed from the femur with minimal essential medium (αMEM without ascorbic acid) and the red blood cells were lysed with sterile water. The cells were centrifuged, re-suspended in αMEM (without ascorbic acid) containing 20% FBS, 1% penicillin-streptomycin, and 2% L-Glutamine, and passed through a cell filter. Non-adherent cells were aspirated 4 days after platting and remaining adherent cells were cultured until 70% confluence before being trypsinized and seeded on various scaffolds.

MTT cell viability assay (Roche) was performed to quantify cell attachment and early proliferation on the electrospun scaffolds. Scaffolds (n=3 per time point) were cut into 6.35-mm diameter circles using a hole punch, sterilized under UV for 1 hr each side, and equilibrated in MSC expansion media (αMEM without ascorbic acid, 20% FBS, 2% L-Glutamine, 1% P/S) at 37° C. overnight. Passage 1 rMSCs (15,625 /cm²) were seeded on the scaffolds placed in ultra-low attachment 96-well plates (Corning) and cultured in expansion media for 24 or 96 hr. The MTT assay was performed according to manufacturer's instructions. Absorbance of the MTT product was read on a Multiskan FC microplate photometer (Thermo Scientific) at 570 nm with a 690 nm background correction.

### Unstimulated Differentiation of Rat Bone Marrow Stromal Cells

rMSCs were cultured on the scaffolds in expansion media to determine the effect of scaffold composition on un-stimulated lineage commitment. Scaffolds were sterilized under UV for 1 h each side and equilibrated in MSC expansion media at 37° C. overnight. The scaffolds were placed in ultra-low attachment 24 well plates (Corning) and seeded with passage 1 rMSCs (50,000 /cm²). Following 7, 14, or 21 days in culture, total RNA from the MSCs adhered on the scaffolds and from P0 rMSCs (prior to seeding on scaffolds, time 0) was isolated using TRIzol (Invitrogen) and purified by Direct-Zol miniprep (Zymo Research). RNA was reverse transcribed into cDNA with SuperScript III Reverse Transcriptase (Invitrogen) according to manufacturer's instructions on a GeneAmp 2700 PCR system (Applied Biosystems). qPCR was performed on an Applied Biosystems 7500 Fast Real-Time PCR system with TaqMan Gene Expression Master Mix (Applied Biosystems) and inventoried TaqMan probes for SOX9, osteocalcin, PPARG, and housekeeping gene GAPDH. All reactions were performed in triplicates and the gene expression was quantified using the delta-delta Ct method. Expression data was normalized using GAPDH and plotted as expression relative to rMSCs at time 0.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### Incorporation by Reference

References and citations to other documents, such as patents, patent applications, patent publications, journals, books, papers, web contents, have been made in this disclosure. All such documents are hereby incorporated herein by reference in their entirety for all purposes.

### Equivalents

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance that can be adapted to the practice of this invention in its various embodiments and equivalents thereof.

What is claimed is:

1. A composition comprising hydroxyapatite and a block co-polymer, wherein the block co-polymer comprises hydrophilic blocks and degradable hydrophobic blocks, wherein the composition exhibits hydrophilic surface properties, elasticity and retains mechanical integrity in aqueous environment.

2. The composition of claim 1, wherein the composition possess a stable structural interface between the co-polymer and the hydroxyapatite.

3. The composition of claim 1, wherein the hydroxyapatite is present in a weight percentage of at least 1%.

4-6. (canceled)

7. The composition of claim 1, wherein the block co-polymer comprises blocks of poly(ethylene glycol) and polyesters.

8. The composition of claim 1, wherein the block co-polymer comprises blocks of poly(ethylene glycol) and poly(lactic acid).

9. The composition of claim 1, characterized by the properties of biodegradability, aqueous stability and eletrospinability.

10. The composition of claim 1, wherein the block co-polymer is electrospun into fibrous composite mesh.

11. The composition of claim 1, wherein the block co-polymer is crosslinked forming a three-dimensional polymer-hydroxyapatite network.

12. The composition of claim 1, wherein the composition is a three-dimensional network prepared rapid prototyping.

13. The composition of claim 1, wherein the composition is characterized by a shape-memory property.

14. An article of manufacture made from a composition of claim 1.

15. A medical implant comprising a composition comprising hydroxyapatite and a block co-polymer, wherein the block co-polymer comprises hydrophilic blocks and degradable hydrophobic blocks.

16. The medical implant of claim 15, wherein the implant is a 3-dimensional filler for bony defects, cartilage defects or osteochondral defects.

17. The medical implant of claim 15, wherein the implant is a degradable fibrous membrane wrapped around one or more structural allografts or one or more 3-dimensional synthetic scaffolds to augment tissue repair function.

18. The medical implant of claim 15, wherein the implant is a repair material for bone, cartilage, osteochondral, tendon or ligament damage.

19. The medical implant of claim 15, wherein the implant is biodegradable.

20. The medical implant of claim 15, wherein the implant is capable of supporting attachment of cells.

21. The medical implant of claim 15, wherein the implant is capable of supporting attachment of a biological agent.

22-23. (canceled)

24. A biodegradable composite scaffold prepared from a fibrous composite mesh electrospun from a suspension of hydroxyapatite with an amphiphilic block poly(ethylene glycol-co-lactic acid).

25. A biodegradable, three-dimensional composite scaffold prepared by crosslinking a suspension of hydroxyapatite with an amphiphilic block poly(ethylene glycol-co-lactic acid).

26. A biodegradable, three-dimensional composite scaffold, prepared by rapid prototyping from a suspension of hydroxyapatite with an amphiphilic block poly(ethylene glycol-co-lactic acid).

27-29. (canceled)
